(19) United States
(12) Patent Application Publication
(10) Pub. No.: US 2017/0067921 A1
(43) Pub. Date: Mar. 9, 2017

(54) ISOTOPIC IDENTIFICATION AND TRACING OF BIOLOGIC PRODUCTS

(71) Applicant: John P. JASPER, Niantic, CT (US)

(72) Inventor: John P. JASPER, Niantic, CT (US)

(21) Appl. No.: 15/308,886

(22) PCT Filed: May 7, 2015
(86) PCT No.: PCT/US2015/029708

§ 371 Date: Nov. 4, 2016

Related U.S. Application Data
(60) Provisional application No. 62/010,819, filed on Jun. 11, 2014.
(60) Provisional application No. 61/990,636, filed on May 8, 2014.

(51) Int. Cl. G01N 33/94; G01N 33/58

(52) U.S. Cl. CPC G01N 33/94; G01N 33/58

(57) ABSTRACT

The present invention relates to stable isotopic identification of biologic products, methods of stable isotopic identification of such biologic products, and stable isotopic methods and systems for correlating biologic products to the processes by which they are made.

### RELATED APPLICATIONS

This application claims priority to U.S. Provisional Patent Application No. 61/990,636 filed on May 8, 2014, and U.S. Provisional Patent Application No. 62/010,819 filed on Jun. 11, 2014, the disclosures of each of which are incorporated by reference herein in their entirety.

### FIELD OF THE INVENTION

The present invention relates to stable isotopic identification of biologic products, methods of stable isotopic identification of such biologic products, and stable isotopic methods and systems for correlating biologic products to the processes by which they are made.

### BACKGROUND OF THE INVENTION

A wide range of biologic products are becoming increasingly important in commerce today. Biologic products are common in pharmaceuticals, foods, consumer, and industrial products. Of particular interest are those biologics that are now used as pharmaceuticals and in the field of personalized medicine. Biologics include for example compounds such as heparin, insulin, and monoclonal antibodies.

The stable isotopic composition of matter is a way to characterize and differentiate one material from another.

Large-scale scientific research involving both radioactive and non-radioactive (i.e. stable) isotopes goes back to the time of the Manhattan Project, which produced the first atomic bombs during World War II. Isotopes—whether stable or radioactive—are forms of the same chemical element having different atomic masses. For example, uranium has an isotopic form with a mass of 235 (uranium-235 or ²³⁵U) and also an isotopic form with a mass of 238 (uranium-238 or ²³⁸U). Although radioactive isotopes can be used in the methods and systems of the present invention, the methods and systems herein are focused primarily on non-radioactive, stable isotopes.

By 1942, isotopes had only been known for about thirty years. Most of the research involving isotopes had been theoretical, relating to determining atomic structures and studying the then-mysterious properties of radioactivity. The Manhattan Project changed all that. The dire urgency of the war effort led to the development of sophisticated techniques for separating and identifying isotopes. One of these techniques, Isotope Ratio Mass Spectrometry (or IRMS for short), which is used to measure the relative abundance of isotopes in a sample, is now an important tool for studying and using isotopes.

Furthermore, the stable isotopic composition of matter has been recognized since about 1945 as a criterion for highly-specifically differentiating one material from another with the same elemental composition. In the field of geochemical oil exploration and prospecting, measurement of the isotopic compositions of large numbers of individual organic compounds of oil samples from various oil reservoirs have assisted in clarifying the origin of specific compounds correlating the organic compounds with particular petroleum sources, recognizing the existence of multiple petroleum sources, examining the mechanisms of petroleum generation, source mixing, and improving the sensitivity of petroleum migration studies. This information, particularly in connection with seismological data, can be used to predict locations of other oil reservoirs to which oil may have migrated from a common source of generation or formation.

Isotope ratio monitoring has had further applications in the biomedical field, wherein non-radioactive and stable isotopes are used as tracer labels in drug metabolism and other biomedical studies where natural variations in isotopic abundances may also carry additional information regarding sources and fates of metabolites. Additionally, radioactive and stable isotopic labeling apparatus and methods in the medical fields employ typically costly labeled compounds having isotope ratios much different than those found in natural abundance.

Methods and systems for isotope identification are described in U.S. Pat. No. 7,323,341 B1, to Jasper, issued Jan. 29, 2008 and U.S. Pat. No. 8,367,414 B2, to Jasper, issued Feb. 5, 2013, which are incorporated by reference herein in their entirety. U.S. Pat. No. 7,323,341 B1 states that it relates to a stable isotopic identification and method for identifying products using naturally occurring isotopic concentrations or isotopic ratios in products, especially in the pharmaceutical industry, and more particularly to an identification and a method utilizing such isotopic concentrations or ratios in a machine readable form for identifying products and tracking products through manufacturing, marketing and use of a product, and readily indexing product information to the product. U.S. Pat. No. 8,367,414 B2 states that it relates to the field of isotope analysis and, in particular, an emerging new field of analytical chemistry that is directed to the derivation of information regarding the origins of synthetic products from processes in which the amounts or ratios of isotopes in either synthetic starting materials, intermediates or products are traced.

Methods for characterizing manufacturing pathways using isotopic methods have also been described. See, e.g., J. P. Jasper, L. E. Weaner, and J. M. Myers, Process Patent Protection: Characterizing Synthetic Pathways by Stable-Isotope Measurements, Pharmaceutical Technology, 2007, 31(3):68-73, which is incorporated by reference herein in its entirety. This reference describes methods by which precise analyses of stable-isotopic abundances can be used in security and forensic applications for pharmaceutical materials. These methods include product and process authentication of raw materials, pharmaceutical intermediates, drug substances, formulated drug products, and synthetic pathways. Since the inception of these techniques, there have been yet further improvements in isotope ratio monitoring sensitivity and precision, as well as frequency, and a reduction in sample size requirements.

Isotope identification methods and systems would be useful for biologic products, which present special challenges compared to lower molecular weight organic compounds, i.e. typically those of about 1000 molecular weight or less.

In many instances, it would be highly desirable to have isotope methods and systems for biologic products and processes, including those used in or as active pharmaceutical ingredients (APIs).

It is apparent from the above there is an ongoing need for new methods and systems which could be applied to biologics.

### BRIEF DESCRIPTION OF THE FIGURES

FIG. 1 depicts the typical structure of heparins (viz., relative elemental composition of C₁₂H₁₅NO₁₉S₃Na₄; molecular weight: 10-15 kDa). (AT—antithrombin)

FIG. 2 depicts the isotopic composition of a reaction product plotted as a function of reaction yield. The isotopic composition, δ, increases as the reaction yield approaches 1, i.e. as the reaction approaches completion. The symbol, ‰, designates permil or what is also referred to as parts per thousand.

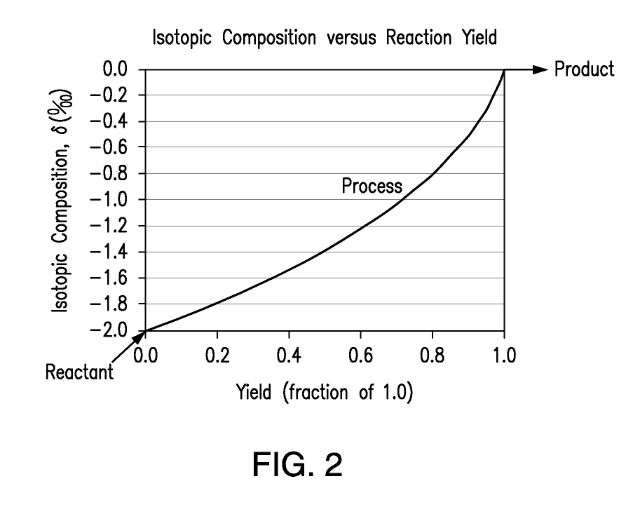

FIG. 3 depicts the carbon-isotopic composition (δ¹³C ‰ vs VPDB) of synthetic intermediates (C, E, G) and the final product (I) for a hypothetical process as a function of reaction step with (upper dotted line) and without (lower solid line) the contributions of partial-reaction completion (f) and isotopic fractionation (ε). (VPDB—Vienna Pee Dee Belemnite)

FIG. 4 depicts the carbon-isotopic differences between isotopic compositions predicted and those which would be observed in the absence of isotope effects, δ_{P}*−δ_{P}, ‰, corresponding to the values in Table 1 as presented in the patent application.

FIG. 5 depicts a principle component analysis (PCA) of the stable isotopic composition of heparin samples: biplot of scores and loadings. The five heparin samples (avg 1/Porcine heparin, 2/Bovine heparin, 3/Ovine heparin, 4/Porcine heparin, & 5/Porcine heparin; avg—average of three sample replicates to estimate intrasample variation in the data set) are indicated by the ovals. The correlation between the stable isotopes can be deduced from the loadings, as well as the ability for each of the five stable isotopes to differentiate between the heparin samples.

FIG. 6 depicts an isotopic bivariate (δ¹³C ‰ vs VPDB-δ¹⁸O ‰ vs VSMOW) plot of three (ovine, bovine, porcine) heparin samples. This is a graph for source differentiation as it shows the separation of USA-porcine heparin from China-sourced porcine heparin and Spain-sourced porcine heparin. Additionally it shows a significant differentiation of the three types of heparin (porcine, ovine, and bovine). (VPDB—Vienna Pee Dee Belemnite, VSMOW—Vienna Standard Mean Ocean Water)

FIG. 7 depicts an isotopic bivariate (δD ‰ vs VSMOW-δ¹⁸O ‰ vs VSMOW) plot of three (ovine, bovine, porcine) heparin samples. This graph demonstrates the maximum resolving power for the three types of heparin. (VSMOW—Vienna Standard Mean Ocean Water)

FIG. 8 depicts an isotopic bivariate (δ¹⁵N ‰ vs Air-δ¹⁸O ‰ vs VSMOW) plot of the (ovine, bovine, porcine) heparin samples. This figure strongly resolves the three types of heparin and along with FIG. 9 demonstrates a close similarity of δ¹⁵N and δ³⁴S. (VSMOW—Vienna Standard Mean Ocean Water)

FIG. 9 depicts an isotopic bivariate (δ³⁴S‰ vs VCDT-δ¹⁸O ‰ vs VSMOW) plot of the (ovine, bovine, porcine) heparin samples. This figure strongly resolves the three types of heparin and along with FIG. 8 demonstrates a close similarity of δ¹⁵N and δ³⁴S. (VCDT—Vienna Canyon Diablo Troilite, VSMOW—Vienna Standard Mean Ocean Water)

### SUMMARY OF THE INVENTION

The present invention relates to stable isotopic identification of biologic products, methods of stable isotopic identification of such biologic products, and stable isotopic methods and systems for correlating biologic products to the processes by which they are made.

### DETAILED DESCRIPTION OF THE INVENTION

The present invention relates to a method for objectively identifying or objectively characterizing a biological product, comprising: obtaining isotopic data, such as isotope ratios, from elements present in said biological product; providing a mathematical array that includes the isotopic data, the mathematical array being fixed in a readable form, said readable form with said mathematical array fixed thereon being an identification of said biological product; and wherein the isotopic data does not include data obtained from a taggant.

In one aspect the present invention relates to a method for objectively identifying or objectively characterizing a biological product, comprising: obtaining isotopic data from elements present in said biological product; providing a mathematical array that includes the isotopic data, the mathematical array being fixed in a readable form, said readable form with said mathematical array fixed thereon being an identification of said biological product; for example, wherein the isotopic data comprises isotopic data for at least one isotope of an element selected and from the group consisting of carbon, hydrogen, nitrogen, oxygen, and sulfur.

In another aspect the present invention relates to a method for constructing an isotopic process profile for a biological product made using a known synthetic process, comprising: obtaining a first isotopic composition profile for elements present in the biological product and a second isotopic composition profile for elements present in one or more starting materials used to make the biological product; determining isotopic fractionation values for one or more reaction steps in the known synthetic process; and providing a database that includes a plurality of data selected from the group consisting of (i) the first isotopic composition profile for the biological product, (ii) the second isotopic composition profile for one or more starting materials used to make the biological product, and (iii) isotopic fractionation values for one or more reaction steps in the known synthetic process; wherein the database is an isotopic process profile of the biological product.

In another aspect the present invention relates to a method for determining whether a biological product of undefined origin was made by a (e.g., first) known synthetic process, comprising: obtaining a first isotopic composition profile for elements present in the biological product; providing fractionation information regarding the first known synthetic process, the starting materials used to make the biological product, or both; and inferentially determining whether the biological product of undefined origin was made by the first known synthetic process by comparing the first isotopic composition profile to the information.

In another aspect the present invention relates to a method for monitoring process quality of a biological synthesis process for a biological product, comprising: defining an acceptable range of isotopic abundance for elements present in the biological product at an intermediate point or an end point in the biological synthesis process for at least one stable isotope, the acceptable range encompassing isotopic abundance values that exist when the process is proceeding in an acceptable manner; periodically extracting samples from the biological synthesis process at the intermediate point or the end point; measuring the actual isotopic abundance for the at least one stable isotope in the samples; and comparing the actual isotopic abundance to the acceptable range to determine whether the biological synthesis process is proceeding in an acceptable manner.

In another aspect the present invention relates to a system for monitoring process quality of a biological synthesis process for a biological product, comprising: a sample extraction device operable to periodically obtain samples from a process stream for the biological synthesis process at an intermediate point or an end point in the process; a measuring instrument operable to receive the samples from the extraction device and determine actual isotopic abundance information for one or more isotopes for elements in the samples; and a computer processor operable to store and display the isotopic abundance information.

In another aspect the present invention relates to a method for making a new biological product batch for a biological product that has a highly-specific or unique isotopic composition profile for elements present in the biological product different than a previously-made biological product batch with the same molecular content, comprising adjusting at least one aspect of the manufacturing process for the biological product in a manner selected from the group consisting of (i) selecting a starting material having a different isotopic composition profile for elements present in the starting material, (ii) identifying a chemical reaction in the process that has an isotope effect, and halting the reaction at a different stage short of completion, (iii) identifying a chemical reaction in the process that has an isotope effect, and making the limiting reagent one that is not used to derive the isotopic composition profile of the biological product, (iv) altering the amount of the limiting reagent that is available for reaction, and (v) mixing into the product an excipient having a different isotopic composition profile for elements present in the excipient.

In another aspect the present invention relates to a method or system wherein the biological product has a molecular weight of at least about 1000 daltons.

In another aspect the present invention relates to a method or system wherein the biological product has a molecular weight of at least about 1000 daltons up to about 2,000,000 daltons.

In another aspect the present invention relates to a method or system wherein the biological product has a molecular weight of at least about 1000 daltons up to about 1,000,000 daltons.

In another aspect the present invention relates to a method or system wherein the biological product has a molecular weight of at least about 1000 daltons up to about 500,000 daltons.

In another aspect the present invention relates to a method or system wherein the biological product has a molecular weight of at least about 1000 daltons to about 100,000 daltons.

In another aspect the present invention relates to a method or system wherein the biological product is selected from abciximab (ReoPro), adalimumab (Humira), ado-trastuzumab emtansine (Kadcyla), alemtuzumab (Campath-1H; Lemtrada; MabCampath), alirocumab (in clinical trials), basiliximab (Simulect), belimumab (Benlysta), bevacizumab (Avastin), blinatumomab (Blincyto), bococizumab (PF-04950615—in clinical trials), brentuximab vedotin (Adcetris), canakinumab (Ilaris), catumaxomab (Removab), certolizumab pegol (Cimzia), cetuximab (Erbitux), daclizumab (Zenapax), Dekavil (Pfizer—in clinical trials), denosumab (Prolia), dinutuximab (Unituxin—in clinical trials), eculizumab (Soliris), efalizumab (Raptiva), emactuzumab (Lemtrada—in clinical trials), etanercept (Enbrel), etrolizumab (RG7413—in clinical trials), evolocumab (in clinical trials), gantenerumab (RG1450—in clinical trials), gemtuzumab ozogamicin (Mylotarg), golimumab (Simponi), heparin (Lipo-Hepin/Liquaemin/Panheparin), ibritumomab tiuxetan (Zevalin), infliximab (Remicade), inotuzumab (ozogamicin—in clinical trials), insulin glargine (Lantus), interferon beta-1a (Avonex), ipilimumab (Yervoy), lampalizumab (RG7417—in clinical trials), lebrikizumab (RG3637—in clinical trials), lifastuzumab vedontin (RG7599—in clinical trials), mepolizumab (in clinical trials), motavizumab (Numax), muronomab-CD3 (Orthoclone OKT3), natalizumab (Tysabri), necitumumab (in clinical trials), nivolumab (Obdivo), obinutuzumab (Gazyva—in clinical trials), ocrelizumab (RG1594—in clinical trials), ofatumumab (Arzerra), omalizumab (Xolair), palivizumab (Synagis), panitumumab (Vectibix), PD-0360324 (Pfizer—in clinical trials), pegfilgrastim (Neulasta), pembrolizumab (Keytruda), pertuzumab (Perjeta—in clinical trials), PF-03446962 (Pfizer—in clinical trials), PF-04236921 (Pfizer—in clinical trials), PF-05082566 (Pfizer—in clinical trials), PF-05230907 (Pfizer—in clinical trials), PF-05236812 (AAV-003—in clinical trials), PF-05280602 (Pfizer—in clinical trials), PF-05285401 (Pfizer—in clinical trials), PF-06252616 (Pfizer—in clinical trials), PF-06263507 (Pfizer—in clinical trials), PF-06342674 (Pfizer—in clinical trials), PF-06480605 (Pfizer—in clinical trials), PF-06647263 (Pfizer—in clinical trials), PF-06650808 (Pfizer—in clinical trials), PF-00547659 (Pfizer—in clinical trials), polatuzumab vedotin(RG7596—in clinical trials), ponezumab (PF-04360365—in clinical trials), ramucirumab (Cyramza), ranibizumab (Lucentis), raxibacumab (ABThrax), rituximab (Rituxan; MabThera), secukinumab (Cosentyx), siltuximab (Sylvant), Tanezumab (Pfizer—in clinical trials), tocilizumab (Actemra—in clinical trials), tositumomab-I-131 (Bexxar), trastuzumab (Herceptin), ustekinumab (Stelara), vanucizumab (RG7221—in clinical trials), vedolizumab (Entyvio), and combinations thereof.

In another aspect the present invention relates to a method or system wherein the biological product is heparin.

In another aspect the present invention relates to a method or system wherein the heparin is mammalian heparin.

In another aspect the present invention relates to a method or system wherein the mammalian heparin is selected from human heparin, bovine heparin, ovine heparin, porcine heparin, and mixtures thereof.

In another aspect the present invention relates to a method or system wherein the mammalian heparin is human heparin.

In another aspect the present invention relates to a method or system wherein the mammalian heparin is bovine heparin.

In another aspect the present invention relates to a method or system wherein the mammalian heparin is ovine heparin.

In another aspect the present invention relates to a method or system wherein the mammalian heparin is porcine heparin.

In another aspect the present invention relates to a method or system wherein the elements are selected from elements that have two or more isotopes.

In another aspect the present invention relates to a method or system wherein the elements are selected from hydrogen, carbon, nitrogen, oxygen, sulfur, chlorine, bromine, and combinations thereof.

In another aspect the present invention relates to a method or system wherein the isotopes are stable isotopes.

In another aspect the present invention relates to a method or system where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In another aspect the present invention relates to a method or system wherein isotope ratios are selected from the stable isotopes, for example, the isotope ratios are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

In another aspect the present invention relates to a method or system wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

In another aspect the present invention relates to a method or system wherein the isotope ratio is ²H/¹H.

In another aspect the present invention relates to a method or system wherein the isotope ratio is ¹³C/¹²C.

In another aspect the present invention relates to a method or system wherein the isotope ratio is ¹⁵N/¹⁴N.

In another aspect the present invention relates to a method or system wherein the isotope ratio is ¹⁸O/¹⁶O.

In another aspect the present invention relates to a method or system wherein the isotope ratio is ³⁴S/³²S.

In another aspect the present invention relates to a method or system wherein the isotope ratio is ³⁷Cl/³⁵Cl.

In another aspect the present invention relates to a method or system wherein the isotope ratio is ⁸¹Br/⁷⁹Br.

In another aspect the present invention relates to a method or system wherein the isotopic data is intrinsic isotopic data, the isotopic composition profile is an intrinsic isotopic composition profile, or the isotopic abundance information is intrinsic isotopic abundance information, that is, the isotopic data, the isotopic composition profile, or the isotopic abundance information is found within the material or sample.

### DEFINITIONS

As used herein, the following terms have the following meanings unless expressly stated to the contrary:

The term “biological product” or “biologic product” or “biologic” as used herein refers to a biologically-produced medical product, which is commonly referred to as a “biologic”, for short. Examples of biological products include medicinal products such as vaccines, blood, blood components, antibodies such as monoclonal antibodies, including derivatives and antibody fragments, enzymes, proteins, and the like. Biological products also include materials for viral gene therapy for artificially manipulating a virus to include a desired piece of genetic material into a target gene or cell. In general, biological products are produced by biological processes, such as e.g., fermentation, cell cultures, extractions, purifications, and harvesting from biological sources. Biological products are also produced by genetic engineering techniques such as, e.g., recombinant DNA and RNA procedures, polymerase chain reaction (PCR) amplification, and the like. Biological products are also produced by derivatization and modification of natural product sources. Biological products generally are made by biological processes rather than chemical processes. In the present invention, the biological products are generally those having a molecular weight of about 1000 or greater, although as can be seen from this paragraph there are a wide range of molecular weights. Biological products are known having molecular weights of tens of thousands, and even in to the millions. Molecular weights are typically reported in daltons. For example, human insulin has a molecular weight of 5808 daltons, heparin has a molecular weight of about 12,000 to about 15,000 daltons, most human proteins have a molecular weight in the range of about 10,000 to about 100,000 daltons. Monoclonal antibodies, including, but not limited to fully human, chimeric, humanized, murine IgG subtypes and their fragment derivatives also have typical molecular weight ranges of about 10,000 to about 150,000 daltons, however can also have larger molecular weights, for example an IgM antibody can have a weight of about 750,000 daltons. A typical human DNA molecule of 1000 nucleotides has a molecular weight of about 300,000, and the human ribosome has a molecular weight of about 4.3 Mdaltons.

Antibodies typically have a molecular weight of around 150,000 however can range from about 10,000 to more than 750,000 daltons. For example there are antibody fragments that range in size from about 10,000 as in a domain antibody (dAb), to about 28,000 as in a single chain antibody, (such as scFv), to about 50,000 for an Fab fragment or about 100,000 for an Fab′2 antibody fragment, to about 750,000 for an IgM antibody. The vast majority of the therapeutic antibodies used clinically are IgG molecules having a MW of about 150,000. Another example is an IgA antibody typically around 300,000 daltons, usually found in saliva and mucous membranes.

The present invention is applicable to a wide variety of biological products. Biologics are becoming an ever increasingly important class of compounds expected to account for approximately 17‰ f the total global spending in medicines by 2016 (Van Arnum P., (2013) Tracking Growth in Biologics. Pharmaceutical Technology Vol. 37, Issue 2), which is incorporated by reference herein in its entirety. Non-limiting examples of biologics of the present invention [Drug Name (Trade Name)] that have been approved or are currently in development include: abciximab (ReoPro), adalimumab (Humira), ado-trastuzumab emtansine (Kadcyla), alemtuzumab (Campath-1H; Lemtrada; MabCampath), alirocumab (in clinical trials), basiliximab (Simulect), belimumab (Benlysta), bevacizumab (Avastin), blinatumomab (Blincyto), bococizumab (PF-04950615—in clinical trials), brentuximab vedotin (Adcetris), canakinumab Glaris), catumaxomab (Removab), certolizumab pegol (Cimzia), cetuximab (Erbitux), daclizumab (Zenapax), Dekavil (Pfizer—in clinical trials), denosumab (Prolia), dinutuximab (Unituxin—in clinical trials), eculizumab (Soliris), efalizumab (Raptiva), emactuzumab (Lemtrada—in clinical trials), etanercept (Enbrel), etrolizumab (RG7413—in clinical trials), evolocumab (in clinical trials), gantenerumab (RG1450—in clinical trials), gemtuzumab ozogamicin (Mylotarg), golimumab (Simponi), heparin (Lipo-Hepin/Liquaemin/Panheparin), ibritumomab tiuxetan (Zevalin), infliximab (Remicade), inotuzumab (ozogamicin—in clinical trials), insulin glargine (Lantus), interferon beta-1a (Avonex), ipilimumab (Yervoy), lampalizumab (RG7417—in clinical trials), lebrikizumab (RG3637—in clinical trials), lifastuzumab vedontin (RG7599—in clinical trials), mepolizumab (in clinical trials), motavizumab (Numax), muronomab-CD3 (Orthoclone OKT3), natalizumab (Tysabri), necitumumab (in clinical trials), nivolumab (Obdivo), obinutuzumab (Gazyva—in clinical trials), ocrelizumab (RG1594—in clinical trials), ofatumumab (Arzerra), omalizumab (Xolair), palivizumab (Synagis), panitumumab (Vectibix), PD-0360324 (Pfizer—in clinical trials), pegfilgrastim (Neulasta), pembrolizumab (Keytruda), pertuzumab (Perjeta—in clinical trials), PF-03446962 (Pfizer—in clinical trials), PF-04236921 (Pfizer—in clinical trials), PF-05082566 (Pfizer—in clinical trials), PF-05230907 (Pfizer—in clinical trials), PF-05236812 (AAV-003—in clinical trials), PF-05280602 (Pfizer—in clinical trials), PF-05285401 (Pfizer—in clinical trials), PF-06252616 (Pfizer—in clinical trials), PF-06263507 (Pfizer—in clinical trials), PF-06342674 (Pfizer—in clinical trials), PF-06480605 (Pfizer—in clinical trials), PF-06647263 (Pfizer—in clinical trials), PF-06650808 (Pfizer—in clinical trials), PF-00547659 (Pfizer—in clinical trials), polatuzumab vedotin(RG7596—in clinical trials), ponezumab (PF-04360365—in clinical trials), ramucirumab (Cyramza), ranibizumab (Lucentis), raxibacumab (ABThrax), rituximab (Rituxan; MabThera), secukinumab (Cosentyx), siltuximab (Sylvant), Tanezumab (Pfizer—in clinical trials), tocilizumab (Actemra—in clinical trials), tositumomab-I-131 (Bexxar), trastuzumab (Herceptin), ustekinumab (Stelara), vanucizumab (RG7221—in clinical trials), vedolizumab (Entyvio). Combinations of these biologics including but not limited to those used in combination therapy regimens are also useful herein.

Further examples of biologics are described in “Biologics Research Pushing Frontiers of Science With More Than 900 Medicines and Vaccines in Development.” Medicines in Development: Biologics (2013) Pharmaceutical Research and Manufacturers of America. 2013 Report 1-87, which is incorporated by reference herein in its entirety.

The top 10 selling biologics based on 2013 global sales are adalimumab (Humira), infliximab (Remicade), rituximab (Rituxam/MabThera), etanercept (Enbrel), insulin glargine (Lantus), bevacizumab (Avastin), trastuzumab (Herceptin), pegfilgrastim (Neulasta), ranibizumab (Lucentis), and interferon beta-1a (Avonex/Rebif) as described in Lawrence, Stacy & Lahteenmaki, Riku (2014) Public biotech 2013—the numbers. Top-ten-selling biologic drugs of 2013. Nature Biotechnology 32, 626-632 (2014). doi: 10.1038/nbt2949, Stone, Kathlyn. The Top 10 Biologic Drugs. (2013) About: Money: Pharma. http://pharma.about.com/od/SalesandMarketing/tp/The-Top-10-Biologic-Drugs.htm, and Top 8 blockbuster biologicals 2013. (2014) Genetic Engineering News. Generics and Biosimilars Initiative (GaBI) http://www.gabionline.net/Biosimilars/General/Top-8-blockbuster-biologicals-2013. Posted Jun. 6, 2014, the disclosures of each of which are incorporated by reference herein in their entirety.

Additionally, it is appreciated that there are also generic copies of these biologics to which this current technology could also be applicable, such as biosimilars and interchangeables. Non-limiting examples of biosimilars and interchangeables currently in development are trastuzumab (PF-05280014), rituximab (PF-05280586), adalimumab (PF-06410293), infliximab (PF-06438179), and bevacizumab (PF-06439535).

Biologics cover a diverse range of therapeutic areas, including but not limited to, cardiology/vascular diseases, dermatology, endocrinology, gastroenterology, genetic diseases, hematology, hepatology, immunology, infections and infectious diseases, musculoskeletal, nephrology, neurology, oncology, pulmonary/respiratory diseases, rheumatology, and vaccines.

The term “stable isotope” or “stable isotopes” as used herein refers to those isotopes that have never been observed to decay. It is recognized that all isotopes will eventually decay. Some isotopes such as hydrogen-7 (⁷H) and lithium-4 (⁴Li) have half-lives on the order of 10⁻²⁴ seconds, whereas, in contrast, calcium-48 (⁴⁸Ca) and tellurium-148 (¹⁴⁸Te) have half-lives on the order of 10²⁴ years. The stable isotopes useful in the present invention are generally the naturally-occurring stable isotopes of hydrogen, carbon, nitrogen, oxygen, sulfur, chlorine, and bromine. More specifically these naturally-occurring stable isotopes are hydrogen (hydrogen-1 or ¹H), deuterium (hydrogen-2 or ²H), carbon-12 (¹²C), carbon-13 (¹³C), nitrogen-14 (¹⁴N), nitrogen-15 (¹⁵N), oxygen-16 (¹⁶O), oxygen-18 (¹⁸O), sulfur-32 (³²S), sulfur-34 (³⁴S), chlorine-35 (³⁵Cl), chlorine-37 (³⁷Cl), bromine-79 (⁷⁹Br), and bromine-81 (⁸¹Br).

The symbol, ‰, designates permil or what is also referred to as parts per thousand.

### Heparin

Heparin, which is also known as unfractionated heparin is a glycosaminoglycan. It is a highly sulfated compound and has a very large negative charge density. Heparin is widely used as an injectable anticoagulant. It is also used to prevent coagulation in medical devices, equipment, and assays. For example, it is used to prevent coagulation in renal dialysis equipment.

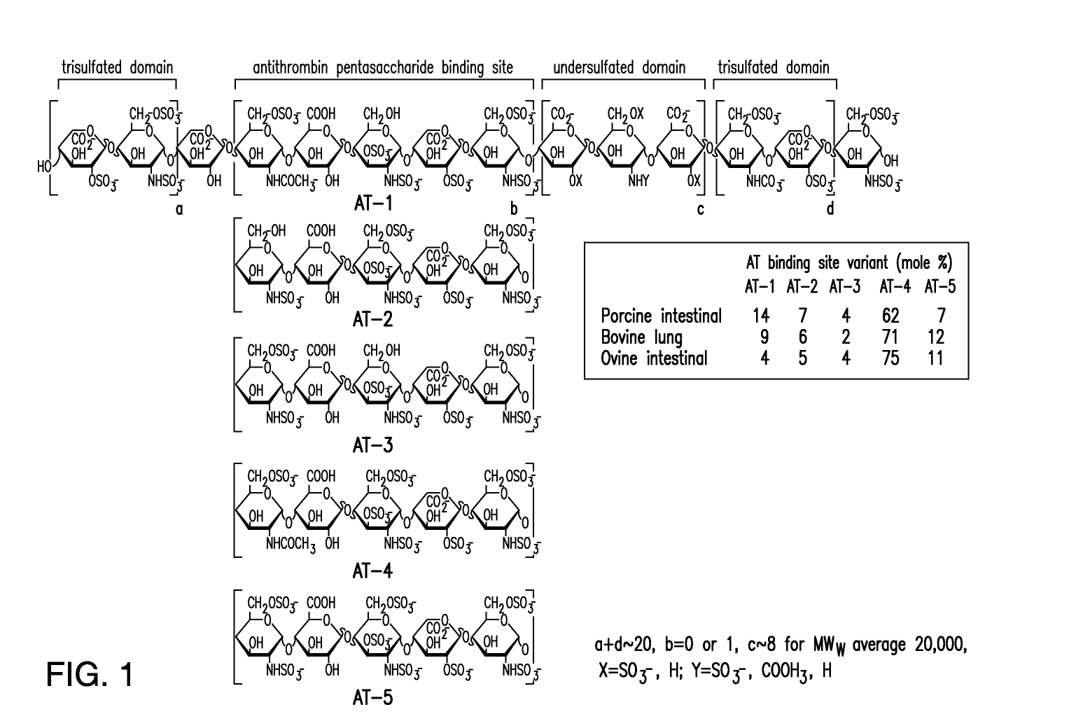

Heparin is found in a wide array of organisms including mammals, birds, and invertebrates. Heparin generally has a molecular weight of about 12,000 to about 15,000 daltons (See FIG. 1). Of particular interest herein are mammalian heparins, with those selected from human, bovine, ovine, and porcine being of special interest. The structural variability of the antithrombin III (AT) binding sites of these mammalian heparins has been previously determining (Fu L, Li G, Yang B, Onishi A, Li L, Sun P, Zhang F, Linhardt R J. 2013. Structural characterization of pharmaceutical heparins prepared from different animal tissues. J Pharm Sci 102:1447-1457)

Methods, systems, and results for stable isotopic analysis of porcine, bovine, and ovine heparins are described in Jasper. J. P., Zhang F., Poe R. B., Linhardt R. J. (2015) Stable Isotopic Analysis of Porcine, Bovine, and Ovine Heparins. Journal of Pharmaceutical Sciences. 104:457-463, which are incorporated by reference herein in their entirety. In this reference, Jasper et al. states that the assessment of provenance of heparin, along with all biologics, is becoming a major concern for the pharmaceutical industry and its regulatory bodies. In this reference, bath-specific stable isotopic compositions of five different animal-derived heparins were performed and the measurements readily allowed their differentiation into groups and/or subgroups based on their isotopic provenance. These measurements revealed a well-defined relationship for source differentiation based on mammalian location and hydrologic environmental isotopes of water. This reference exemplifies the field of isotope analysis and in particular an emerging new field of analytical chemistry that is directed to the derivation of information regarding the origins of complex products such as biologics.

### Stable Isotope Identification Methods

Measurements of the abundances of naturally occurring stable isotopes in pharmaceutical materials can be used to quantitatively characterize both the sources of the products and the synthetic processes used to produce them, as well as the progress of those processes. The methods and systems of the present invention utilize isotopic information for one or more isotope ratios from elements present in samples from the chemical or biological processes of interest. Methods and systems for isotope identification are described in U.S. Pat. No. 7,323,341 B1, to Jasper, issued Jan. 29, 2008; U.S. Pat. No. 8,367,414 B2, to Jasper, issued Feb. 5, 201; and J. P. Jasper, L. E. Weaner, and J. M. Myers, Process Patent Protection: Characterizing Synthetic Pathways by Stable-Isotope Measurements, Pharmaceutical Technology, 2007, 31(3):68-73; which are incorporated by reference herein in their entirety.

For many products, e.g. such as a pharmaceutical product, the source of each atom is known in detail. For example, a methyl carbon will derive from a particular synthetic reactant, an amino nitrogen from another, etc. The measured carbon or nitrogen isotopic composition of the final product will be the weighted average of all carbon or nitrogen positions within the molecule. In turn, this measured isotopic composition will be equal to the weighted average of the isotopic compositions at the precursor positions in the synthetic reacts as modified by generally only two factors: (i) if the synthetic reactions are non-quantitative, any isotope effects which modulate the transfer of material from reactant to products and (ii) in some cases, exchanges of isotopes between products and reaction media.

Isotopic calculations are based on two systems of equations. The first employs mass balances and the second involves integrated forms of rate equations that pertain to kinetically controlled isotopic fractionations. Equations describing mass balances are generally exact when cast in terms of fractional abundances [e.g., ¹³C/(¹²C+¹³C)]. In contrast, assessments of differential rates are based on isotope ratios (e.g., ¹³C/¹²C). When these systems are blended, either approximations or equations with multiple terms are employed. For details, see Hayes J M, 1004; http://www.nosmas.whoi.edu/docs/IsoCalcs.pdf, which is incorporated by reference herein in its entirety.

The relevant isotopic parameters are stoichiometry (n), isotopic abundance (δ), the magnitude of the isotopic effect (ε), and a variable related to conversion of reactants to products (f).

The symbol, n represents the stoichiometry of the reaction, more specifically the number of atoms of a given element (e.g., carbon) in a given molecule involved in the reaction.

As mentioned above, (δ), is a measure of isotopic abundance, and δ is usually reported as the difference in parts per thousand, or permil (‰), from an international standard. δ can be negative or positive depending on whether the sample is enriched or depleted in the heavy isotope relative to the standard. For example, in the case of carbon the difference is calculated as

δ¹³C(‰)=([(Rₛₘₚₗ)/(R_{std})]−1)·(1000) (equation 1)

where Rₛₘₚₗ is the ¹³C/¹²C ratio of the sample and R_{std} is the ¹³C/¹²C ratio in the standard. δ is thus linearly proportional to the isotopic ratio in the sample. Standards are available from the International Atomic Energy Authority and a standard for each isotope is used to determine the zero point of an abundance scale for that isotope. Standards include a particular seawater sample for H and O, calcium carbonate for C, air for N, and a meteorite for S. When the sample is depleted in the heavy isotope relative to the standard, δ is negative and when the sample is enriched it has a positive value. If it has the same isotopic abundance then δ=0. See, J P Jasper, The Increasing Use of Stable Isotopes in the Pharmaceutical Industry, Pharm. Tech., 1999, 23(10):106-114, which is incorporated by reference herein in its entirety.

The magnitude of an isotope effect, (ε), is such that its value depends on details of the reaction and on the relative mass difference between isotopes. Effects are largest for D (deuterium) vs. H and smaller for heavier elements. In general, the values of ε are specific to individual positions within the molecules involved. The isotope effects are largest at the reaction site, much smaller at neighboring positions, and usually not measurable elsewhere. Like δ, ε relates to the isotopic difference between two materials (e. g., reactant and product A and B) and is usually expressed in permil or parts per thousand. For example, for kinetic isotope effects, in the methods and systems employed here, ε=−10‰ means that a reaction site bearing the heavy isotope reacts 10 parts per thousand, or 1%, more slowly than a site bearing a light isotope. For equilibrium isotope effects, ε_{A/B}=15‰ would mean that, at equilibrium, A Is enriched in the heavy isotope by 15 parts per thousand relative to B. Here, A and B refer to specific atomic positions that can be related by a chemical equilibrium.

f is a measure of the progress of a reaction. It is generally the most important variable governing fractionations caused by isotope effects. Its value ranges from 1 to 0 and depends on factors such as temperature, pressure, or availability of reactants. In equilibria (A⇄B), f indicates the position of the equilibrium, with f_{B}=1 indicating complete conversion to B and, at any position, f_{A}+f_{B}=1. In irreversible reactions, f_{X} indicates the portion of reactant X which remains unconsumed, with f_{X}→0 as the reaction proceeds to completion.

The precision of isotopic analyses is typically calculated by two methods. Pooled standard deviations of raw data are typically computed from sets of duplicate or triplicate measurements. From those pooled standard deviations, standard deviations of mean values pertaining to specific substances are calculated. More specifically, the standard deviation of a mean value is the pooled standard deviation divided by n^{1/2}, where n is the number of measurements performed on a given sample. See Jasper, J P, Quantitative estimates of precision for molecular isotopic measurements. Rap. Comm. Mass Spec., 2001 15:1554-1557, which is incorporated by reference herein in its entirety. For carbon, nitrogen, oxygen, and sulfur, the resulting 95% confidence intervals for a result are typically in the range of ±0.1- to ±0.4‰. For hydrogen, the 95% confidence interval is typically ±3‰.

Precise quantitation of stable isotopic compositions in pharmaceutical intermediates and products requires both mass balance and isotopic fractionation equations that are applicable to both single and multi-step reaction sequences. One starts from the most basic requirement of mass balance then considers isotopic fractionations in a single reaction.

### Mass Balance

For A+B→C, where reactants A and B are quantitatively converted to product C, two mass balances can be written:

m_{A}+m_{B}=m_{C} equation (2)

m_{A}δ_{A}+m_{B}δ_{B}=m_{C}δ_{C} equation (3)

where, m_{A}, m_{B}, and m_{C} are molar amounts of carbon (or any other element) in A, B, and C and the isotopic compositions of that carbon (or any other element) in A, B, and C are given by δ_{A}, δ_{B}, and δ_{C}. Equation 2 is a mass balance (i.e., carbon in=carbon out) while equation 3 is an isotopic mass balance (¹³C in=¹³C out). Under the conditions postulated (quantitative conversion) the isotopic composition of C can be computed from those of A and B. See, Hayes J M, 1004; http://www.nosmas.whoi.edu/docs/IsoCalcs.pdf and JP Jasper, The Increasing Use of Stable Isotopes in the Pharmaceutical Industry, Pharm. Tech., 1999, 23(10):106-114, which are incorporated by reference herein in their entirety.

### Isotopic Fractionation

For isotopic fractionations, calculations should take into account factors such as reaction completeness and isotope effects, as these will cause the isotopic composition of C to differ from that computed using the mass balance equation and assuming quantitative conversion of reactants to products. To provide a concrete example, assume that A is present in excess while B, the limiting reactant, is quantitatively converted to product. In that case

n_{A}(δ_{A}−Δ_{A})+n_{B}δ_{B}=n_{C}δ_{C} equation (4)

where n_{A}, n_{B}, and n_{C} represent the numbers of atoms of carbon (or any other element of interest) in A, B, and C. Because A is not quantitatively converted to product, the isotopic compositions of the A-derived positions in C can differ from those in the initial reactant. Here, that isotopic offset is expressed as Δ_{A}, where its value depends on the isotope effect(s) and on the fraction of A that remains unconsumed. If the reaction conditions, particularly the magnitude of the excess of A, are consistent, Δ_{A} will be constant. Because the n values are known exactly, Δ_{A} can be determined from equation 4 after isotopic analysis of the reactants and product (i.e., determination of δ_{A}, δ_{B}, and δ_{C}).

Values of δ_{A}, δ_{B}, and δ_{C} generally do not affect the values of Δ_{A}. Accordingly, once Δ_{A} is known for a given reaction and set of conditions, it is usually necessary only to know two of the δ values in order to compute the third. Thus, for example, when δ_{A}, δ_{A}, and δ_{B}, are known, the isotopic value of the product (δ_{C}) can be calculated.

If neither A nor B is completely consumed during the course of the reaction, and if the rate of the chemical reaction (or position of the chemical equilibrium) is sensitive to isotopic substitution on both reactants, it will be necessary to consider values of both Δ_{A} and Δ_{B}:

n_{A}(δ_{A}−Δ_{A})+n_{B}(δ_{B}−Δ_{B})=n_{C}δ_{C} equation (5)

If reaction conditions cannot be manipulated so that f_{A} and f_{B} (and thus Δ_{A} and Δ_{B}) can be independently driven to completion (i.e., zero), it will be possible to determine only the sum, n_{A} δ_{A}+n_{B} δ_{B}. From theoretical considerations, Δ_{A} and Δ_{B} can be evaluated separately for all values of f_{A} and f_{B} if the isotope effects are known. See, Scott, K M, Lu, X, Cavanaugh, C M, and Liu, J S, Geochim. Cosmochim. Acta, 2004; 68(3):433, which is incorporated by reference herein in its entirety.

Of course, isotopic fractionations like those discussed above accumulate during the different steps of a multi-step synthesis scheme. They can, however, be individually and systematically differentiated, not only for multiple reactants but also for multiple isotopes. To provide an example consider carbon-isotopic fractionations in a hypothetical four-step sequence:

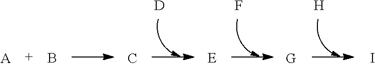

Illustrative carbon skeletons for reactants and products are shown below with pertinent quantities summarized in Table 1.

Illustrative Carbon Skeletons for Reactants and Products

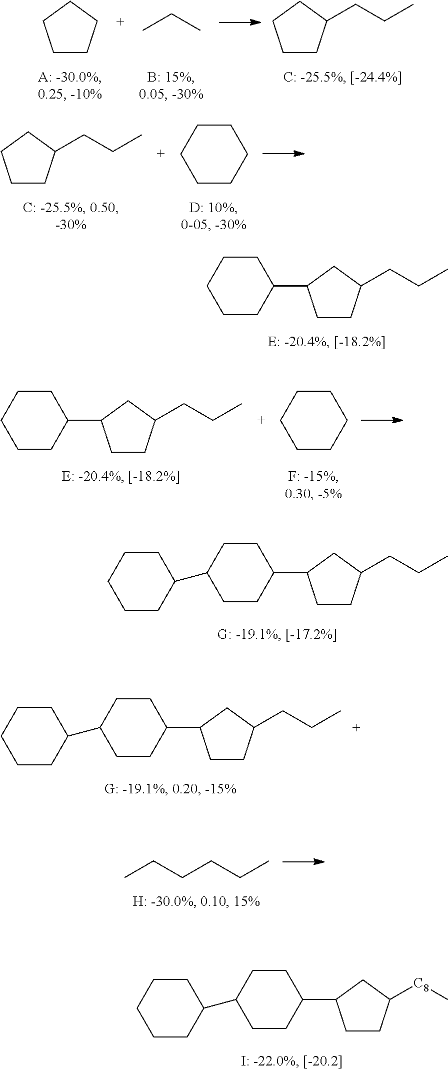

Carbon skeletons of reactants and products in a hypothetical four-step synthetic reaction scheme. This example illustrates the effects of the four key isotopic variables (n, δ, f, ε) on the isotopic compositions of the three synthetic intermediates (C, E, G) and of the final product, I (δ_{P}). For all eight reactants (left), the given numerical values are δ, f, and ε. For all four products (right), the numerical values are the isotopic compositions actually observed (δ_{P}) and that expected in the absence of isotope effects and incomplete consumption of reactants ([δ_{P}*]).

TABLE 1
Properties of Four-Step Synthetic Sequence^{a}

| Reactants | | | | | | Conditions | | | | Products | | |
|---|---|---|---|---|---|---|---|---|---|---|---|---|
| 1 | 2 | n₁ | n₂ | δ₁, ‰ | δ₂, ‰ | f₁ | f₂ | Σε₁, ‰ | Σε₂, ‰ | | δ_{P}*, ‰ | δ_{P}, ‰ |
| A | B | 5 | 3 | −30.0 | −15.0 | 0.255 | 0.055 | −10.0 | −30.0 | C | −24.4 | −25.5 |
| C | D | 8 | 6 | −25.5 | −10.0 | 0.50 | 0.05 | −30.0 | −5.0 | E | −18.2 | −20.4 |
| E | F | 14 | 6 | −20.4 | −15.0 | 0.10 | 0.30 | −15.0 | −5.0 | G | −17.2 | −19.1 |
| G | H | 20 | 6 | −19.1 | −30.0 | 0.20 | 0.10 | −15.0 | −15.0 | I | −20.2 | −22.0 |

^{a}The sequence of reactants and products is given by equation 6 in the text. The numbers of carbon atoms and the carbon-isotopic compositions of reactants 1 and 2 in each step are given by n₁, n₂, δ₁, and δ₂. The fractions of each reactant unconsumed in each step are given by f₁ and f₂. The sums of all carbon isotope effects pertaining to each reactant are given by Σε₁ and Σε₂. The isotopic compositions that successive products would have in the absence of isotope effects are given by δ_{P}* and the isotopic compositions actually observed are given by δ_{P}.

The carbon numbers (n₁, n₂), initial isotopic compositions (δ₁, δ₂), fractions of reactants remaining unconsumed (f₁, f₂) and summed isotope effects (Σε₁, Σε₁) are chosen to be representative of a typical synthetic scheme. All isotope effects are assumed to be kinetic. Values of δ_{P}*, the isotopic compositions that would be observed if isotopic fractionations were absent, are calculated using equation 5 with Δ_{A}=Δ_{B}=0; that is, the simple mass balance equations 2-3. Values of δ_{P}, the isotopic compositions that would actually be observed for the successive products, are calculated using exact forms of integrated rate equations. See Scott, K M, Lu, X, Cavanaugh, C M, and Liu, J S, Geochim. Cosmochim. Acta, 2004; 68(3):433, which is incorporated by reference herein in its entirety.

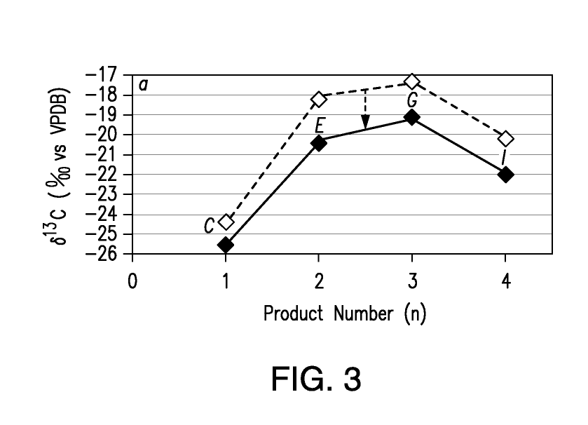

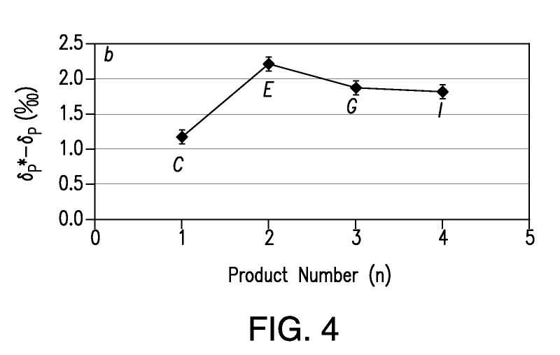

The foregoing illustrates the interplay of the four factors that control the isotopic compositions of manufactured products, namely the stoichiometries and isotopic compositions of the starting materials, isotope effects associated with the synthetic reactions, and the degree to which conversions of precursors to products are quantitative. The isotopic compositions of products are generally dominated by the initial isotopic abundance of the precursor materials and are variously modulated (viz., depleted) by the degree of completion (f) and the magnitude of any isotopic effects (ε, FIG. 3). A plot that summarizes the difference between the isotopic compositions that are predicted and those that would be observed in the absence of isotope effects (δ_{P}*−δ_{P}) is shown in FIG. 4. These values are also shown in the last two columns of Table 1. In the first synthetic step, isotope effects on reactant B are rather large, but that reactant is consumed almost completely. The resulting isotopic fractionation is less than 1‰ (the larger value shown in FIG. 4 pertains to the product and reflects fractions affecting both reactants). In the second step, a large isotope effect and poor conversion of reactant C lead to a large isotopic fractionation at the reaction site. However, fractionation is diluted now that the product contains 14 carbon atoms. As shown in FIG. 4, the overall difference between real and hypothetical unfractionated products is barely doubled. In the remaining steps, where isotope effects are moderate and consumption of reactants is relatively efficient, isotopic generally fractionation declines.

For a chemical or biological process, one can monitor the equilibrium between two isotopes, which are designated as “A” and “B”. Consider the case of a general system in which there are three different atoms or isotopes under consideration:

A+B→P

where A, B, and P contain n_{A}, n_{B}, and n_{P} atoms of the element under consideration. The system can be described by the following equation for determining the progress of the process via the isotopic abundance, δ:

$${δ}_{P} = {δ}_{P}^{*} - \frac{1}{{n}_{P}} [ \frac{{f}_{A}}{1 - {f}_{A}} ln {f}_{A} ( {ɛ}_{A 1} + {ɛ}_{A 2} + … ) + \frac{{f}_{B}}{1 - {f}_{B}} ln {f}_{B} ( {ɛ}_{B 1} + {ɛ}_{B 2} + … ) ] \qquad equation ( 6 )$$

In equation 6, δ_{P} and δ_{P}* are the observed isotopic composition of the product and the idealized isotopic composition of the product, respectively, ε_{A1}, ε_{B2}, etc. are the primary kinetic isotope effects at the reaction sites in A and B, respectively, and ε_{A2}, ε_{B2}, etc. are the secondary isotope effects, and f is a measure of the progress of the reaction.

### Continuous Monitoring Methods

The present invention relates to methods for continuously monitoring the progress of biological processes utilizing isotopic information for one or more isotope ratios from elements present in samples from these processes. These continuous methods utilize the stable isotope identification methods as described herein.

Until recently, it was generally not possible to sample, monitor, or measure a process for isotopic determinations more frequently than say, once about every 15 minutes, and thus not possible to provide methods and systems for continuous isotope information determination. The reason for these relatively long time intervals was due to sampling and instrumentation limitations. In the present invention, it is now possible to sample, monitor, or measure at or over very small time intervals, the result which is for all intents and practical purposes, is perceived as a continuous sampling, monitoring, or measuring of the chemical or biological process.

### Monitoring System

The present invention relates to systems for analyzing or monitoring biological samples or processes utilizing isotopic information for one or more isotope ratios from elements present in samples from these biological systems. The systems of the present invention are useful for carrying out the methods of the present invention. The systems of the present invention comprise the following components, each of which are described in further detail: (a) a device for sampling the process, (b) an interface, (c) an isotope analyzer, and (d) a computerized data system (CDS). In further embodiments, the systems of the present invention can also include a reactor in which the chemical processes or the biological processes of the present invention are conducted or contained.

### Device for Sampling the Process

The systems of the present invention comprise a device for sampling the process.

This device is an instrument or probe that samples materials from the reaction system or vessel. The device can also be a stream of an inert gas which is either bubbled through or passed over the system for removing, i.e. sampling gaseous or volatile products or by-products of the chemical or biological process. An example of such a sampling system can be a tube, hose, or line for delivering a stream of an inert gas, such as helium, and a corresponding tube, hose, or line for collecting the effluent gas and any desired products or by-products for isotope analysis.

### Interface

The systems of the present invention comprise an interface.

The interface is the connector between the sampling device and the isotope analyzer. This interface can take a variety of forms and can be either electronic or mechanical.

### Isotope Analyzer

The systems of the present invention comprise an isotope analyzer.

In theory, a wide range of isotope analyzers can be used in the systems of the present invention. The isotope analyzer is a device for measuring or determining the desired stable isotope ratios of the sampled process. Examples of isotope analyzers useful for the methods and systems of the present invention include those selected from: (a) cavity ring-down spectrometer (CRDS), (b) an isotope ratio mass spectrometer (IRMS), and (c) a nuclear magnetic resonance (nmr) spectrometer.

### Cavity Ring-Down Spectrometer (CRDS)

Cavity ring-down spectroscopy (CRDS) is an optical spectroscopic technique utilizing a cavity ring-down spectrometer (CRDS). The method is highly sensitive, down to the 0.1% level, and is used to measure the light absorption of samples, i.e. the absolute optical extinction, that scatter and absorb light such as gas samples. A common cavity ring-down spectrometer configuration comprises a laser used to illuminate a high-finesse optical cavity, which essentially comprises two highly reflective mirrors. When the laser is in resonance with a cavity mode, the intensity of the laser light builds up in the cavity due to constructive interference. When the laser is turned off, the exponentially decaying light intensity leaking from the cavity is measured. This decaying laser light is reflected between the mirrors many thousands of time giving an effective path length on the order of kilometers.

When a sample is placed in the cavity, such as a sample containing a desired isotope, the intensity of the light decreases faster due to the absorption of the sample. The cavity ring-down spectrometer measures how long it takes for the light to decay, or “ring-down” to lie of its initial intensity both with and without the sample, thus giving a measure of the amount of the sample absorbing the laser light. See, Giel Berden; Rudy Peeters; Gerard Meijer (2000). “Cavity ring-down spectroscopy: Experimental schemes and applications”. International Reviews in Physical Chemistry 19 (4): 565-607; and Paldus, B. A. and Kachanov, A. A., An Historical Overview of Cavity Enhanced Methods (Einstein Centennial Review Article), Canadian Journal of Physics, 83, pp. 975-999 2005 NRC; which are incorporated by reference herein in their entirety.

An example of a cavity ring-down spectrometer useful in the methods and systems of the present invention includes a Picarro CRDS G2131-i Analyzer sold by Picarro Inc., 3105 Patrick Henry Drive, Santa Clara, Calif. 95054.

### Isotope Ratio Mass Spectrometer (IRMS)

Isotope-ratio mass spectrometry (IRMS) is a type of mass spectrometry. The method uses an isotope-ratio mass spectrometer (IRMS) measure the relative abundance of isotopes in a given sample. For the methods and systems of the present invention, isotope-ratio mass spectrometry is used to measure or analyze the isotopic variations of stable isotopes in samples of interest. The isotope-ratio mass spectrometer (IRMS) allows the precise measurement of mixtures of naturally occurring isotopes. See, Townsend, A. (ed.) (1995) Encyclopaedia of Analytical Science Encyclopaedia of Analytical Science. London: Academic Press Limited, which is incorporated by reference herein in its entirety.

Isotope-ratio mass spectrometers useful herein can be of either the magnetic sector design or the quadrupole design, with the magnetic sector design generally being preferable. The magnetic sector type, also known as the “Nier type”, after its designer Alred Nier, operates by ionizing the sample and accelerating it over a potential (usually in the kilo-volt range). The resulting stream of ions is thus separated according to their mass-to-charge ration, or m/z.

See, Goetz, A.; Platzner, I. T. (Itzhak Thomas); Habfast, K.; Walder, A. J. (1997). Modern isotope ratio mass spectrometry. London: J. Wiley, which is incorporated by reference herein in its entirety.

An example of an isotope-ratio mass spectrometer useful herein is a ThermoScientific DELTA V™ Plus Isotope Ratio Mass Spectrometer. See, http://www.thermoscientific.com/en/product/delta-v-plus-isotope-ratio-mass-spectrometer.html, which is incorporated by reference herein in its entirety.

### Nuclear Magnetic Resonance (NMR) Spectrometer

A nuclear resonance (NMR) spectrometer is a very common analytical device that is even now available in many undergraduate chemistry laboratories. NMR spectroscopy is an analytical method that uses the magnetic properties of certain atomic nuclei to provide both qualitative and quantitative physical and chemical properties of atoms and the molecules in which they are contained. When placed in a magnetic field, various nuclei or isotopes, e.g., ¹H and ¹³C, absorb electromagnetic radiation at a frequency characteristic of the isotope. Such information can include structures, dynamics, chemical environment, and also isotope and isotope ratio information.

See V. Govindaraju, K. Young, and A. A. Maudsley, Proton NMR chemical shifts and coupling constant for brain metabolites. NMR in Biomedicine, Volume 13, Issue, pages 129-153, May 2000; and J. H. H. Nelson and J. H. Nelson, Nuclear Magnetic Resonance Spectroscopy: 1^{st} Edition, ISBN-13: 9780130334510, 2002, Prentice Hall, which are incorporated by reference herein in their entirety.

An example of a nuclear magnetic resonance spectrometer useful herein is a Thermo Scientific picoSpin 80 NMR Spectrometer.

### Computerized Data System (CDS)

The systems of the present invention comprise a computerized data system (CDS). A computerized data system is the computer or computer system for collecting, processing, and storing the isotope ratio data generated from the sampling and collection of samples from the processes of the present invention. In many cases, the computerized data system is integrated into or closer associated with the isotope analyzer. In other others it is a separate or stand-alone computer, whether a hand-held, lap-top, desk-top, or main-frame computer which is attached or associated with the isotope analyzer. By associated is meant that the data from the isotope analyzer is either sent electronically, wirelessly, or transmitted via a separate storage device such as a CD or flash-drive.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Stable-Isotopic Analysis of Three Types of Heparin Samples: Porcine, Bovine, and Ovine

A study of the batch-specific [carbon-(δ¹³C), nitrogen-(δ¹⁵N), oxygen-(δ¹⁸O), sulfur-(δ³⁴S), and hydrogen-(δD)] stable-isotopic compositions of five samples of animal-derived heparin was performed to test whether those samples could be either grouped or differentiated by their stable-isotopic compositions. Twenty-five measurements of the δ¹³C, δ¹⁵N, δ¹⁸O, δ³⁴S, and δD values of these samples readily allowed their differentiation of the samples into groups and/or subgroups based on their isotopic provenance. Initial Principal Component Analysis showed results. See, Jasper. J. P., Zhang F., Poe R. B., Linhardt R. J. (2015) Stable Isotopic Analysis of Porcine, Bovine, and Ovine Heparins. Journal of Pharmaceutical Sciences. 104:457-463.

In particular, the stable-isotopic analyses revealed that (i) stable-isotopic measurements of biologic molecules (MW˜10-15 kDa) were feasible; (ii) in bivariate plots, the δ¹³C vs δ¹⁸O plot reveals a well-defined relationship for source differentiation, separating USA-porcine heparin from non-USA heparin; (iii) the δD vs δ¹⁸O plot revealed the most well-defined relationship for source differentiation based on the hydrologic-environmental isotopes of water (D/H and ¹⁸O/¹⁶O) and (iv) the δ¹⁵N vs δ¹⁸O and δ³⁴S vs δ¹⁸O relationships are both very similar, likely reflecting the food sources for the heparin producers.

The stable-isotopic compositions of selected light elements (e.g., C, N, O, S, H) are very useful in determining the natural origin of materials (e.g., Smith and Epstein, 1971; Fogel, 2010) as well as the “manufacturer or batch” origin of the products and materials manufactured from them (e.g., Jasper, 2004; Jasper et al., 2007). Stable carbon isotopes are particularly useful in differentiating large classes of plant organic matter based on their photosynthetic pathways: e.g., C3-terrigenous- vs. C3-algal sources vs. C4-terrigenous sources, where C3 plants fractionate to a greater degree (using Rubisco) than do C4 plants (using RUBP Carboxylase). In addition, the stable-isotopic composition of nitrogen (δ¹⁵N) is highly variable in nature via a number of pathways for inorganic as well as organic species (Fogel, 2010). Further, the stable-isotopic composition of sulfur (δ³⁴S) has been demonstrated to have very high dynamic ranges (observed ranges/1σ standard deviations >200) in proprietary pharmaceutical studies and typically serves as an excellent tracer of isotopic provenance.

By way of background, the stable-isotopic characterization of individual batches of industrial products (“Nature's Fingerprint®”) via their patented synthetic pathways (“Isotopic Pedigree”) provides a highly-specific means to identify product batches and to protect chemical process patents (Jasper et al., 2007). In the period between the expirations of composition-of-matter patents and of their paired process patents, stable-isotopic analyses permit a novel and efficient means by which to protect the intellectual property of bio/pharmaceutical products (Jasper et al., 2007). Since 2007, Jasper and colleagues have provided value for pharmaceuticals and neutraceuticals (Jasper et al., 2012) via the isotopic fractionations (or, differences) in the stable-isotopic compositions of various light elements (Jasper et al., 2005).

This is a study of the isotopic provenance and quantification of the variability of a suite of animal-derived heparin product batches. The isotopic provenance (or, “manufacturer's fingerprint”) should give quantitative indications of the sources of these and preliminary insights into their processes of production.

### Experimental—Samples:

Five samples of animal-derived heparin were provided for this study by the RPI laboratories (Table 2). The heparin was derived from porcine, bovine, and ovine sources. The three porcine heparins were sourced from the United States, China, and Spain. The ovine and bovine heparins emanated from Sigma Inc. (US). Typical relative elemental compositions of these heparins are C₁₂H₁₅NO₁₉S₃Na₄ with molecular weights in the range of 10-15 kDa.

### Estimates of Uncertainty.

The uncertainty (or precision) of the isotopic measurements in this study were estimated via the pooled standard deviations (SD) of recent studies. In those cases, the pooled SD of raw data were estimated to derive a representative standard deviation from the whole raw data set in which small numbers of replicates (viz., n=1-3) were pooled to generate a representative standard deviation of the whole sample suite (Jasper, 2001). Characteristic one sigma (1σ) standard deviations for the isotope values used in this study were: δ¹³C (±0.06‰), δ¹⁵N (±0.06‰), δ¹⁸O (±0.08‰), δD (±1.1‰) and (and δ³⁴S (±0.04‰), as shown in Table 2.

### Results—

The ¹³C, ¹⁸O, ¹⁵N, ³⁴S, and D Analyses of Heparin Samples

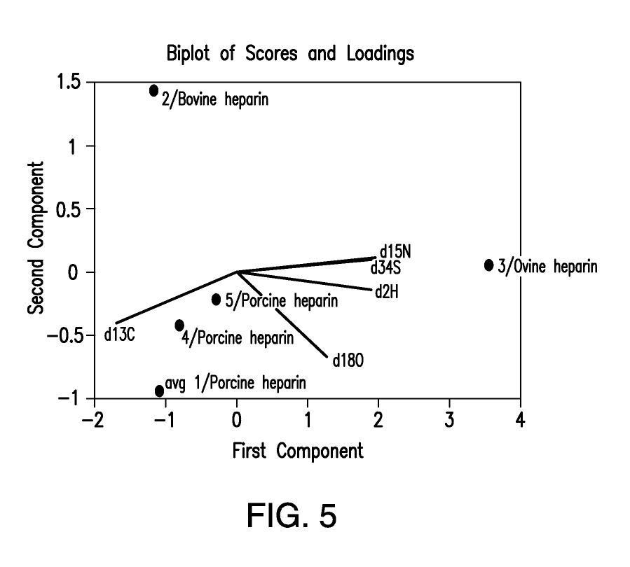

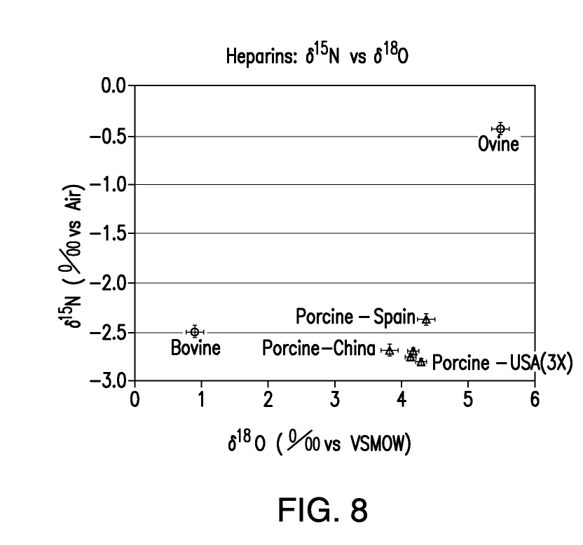

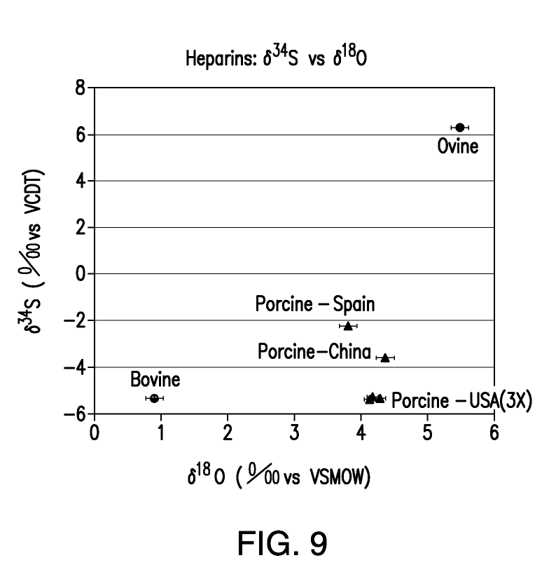

The results of stable-isotopic analyses (δ¹³C, δ¹⁸O, δ¹⁵N, δ³⁴S and δD) of the five heparin samples given are presented in Table 2 and in FIGS. 5-9.

TABLE 2
Stable-Isotopic Compositions of Heparin Samples

| Sample | | d¹³C | PSE ¹³C | d¹⁵N | PSE ¹⁵N | d¹⁸O | PSE ¹⁸O | dD | PSE D | d³⁴S | PSE ³⁴S |
|---|---|---|---|---|---|---|---|---|---|---|---|
| Name | Source | ‰ | ‰ | ‰ | ‰ | ‰ | ‰ | ‰ | ‰ | ‰ | ‰ |
| 1: Porcine | USA | −14.19 | 0.03 | −2.69 | 0.03 | 4.18 | 0.05 | 31.8 | 0.6 | −5.28 | 0.02 |
| heparin-1 | | | | | | | | | | | |
| 1: Porcine | USA | −14.15 | 0.03 | −2.80 | 0.03 | 4.30 | 0.05 | 33.8 | 0.6 | −5.35 | 0.02 |
| heparin-2 | | | | | | | | | | | |
| 1: Porcine | USA | −14.26 | 0.03 | −2.75 | 0.03 | 4.14 | 0.05 | 33.6 | 0.6 | −5.34 | 0.02 |
| heparin-3 | | | | | | | | | | | |
| 2: Bovine | USA- | −21.08 | 0.06 | −2.49 | 0.06 | 0.90 | 0.08 | 16.3 | 1.1 | −3.57 | 0.04 |
| heparin | Sigma | | | | | | | | | | |
| 3: Ovine | USA- | −28.27 | 0.06 | −0.43 | 0.06 | 5.50 | 0.08 | 80.8 | 1.1 | 6.30 | 0.04 |
| heparin | Sigma | | | | | | | | | | |
| 4: Porcine | China | −16.55 | 0.06 | −2.68 | 0.06 | 3.82 | 0.08 | 22.7 | 1.1 | −2.24 | 0.04 |
| heparin | | | | | | | | | | | |
| 5: Porcine | Spain | −21.55 | 0.06 | −2.37 | 0.06 | 4.37 | 0.08 | 23.7 | 1.1 | −3.60 | 0.04 |
| heparin | | | | | | | | | | | |

d—δ;
PSE—Pooled Standard Error

In the following sections, the δ¹³C, δ¹⁸O, δ¹⁵N, δ³⁴S and δD data from Table 2 are displayed in bivariate plots and in Principle Component Analysis (PCA) their relationships discussed and assessed. PCA is a statistical procedure that converts a series of observations with possible correlations into a set of linearly uncorrelated variables. It is most useful in demonstrating the variance within data and revealing an internal structure represented by the whole data set.

Principal Component Analysis of the Stable-Isotopic Composition of Heparin Samples

Bivariate Isotope Plots of the Stable-Isotopic Composition of Heparin Samples

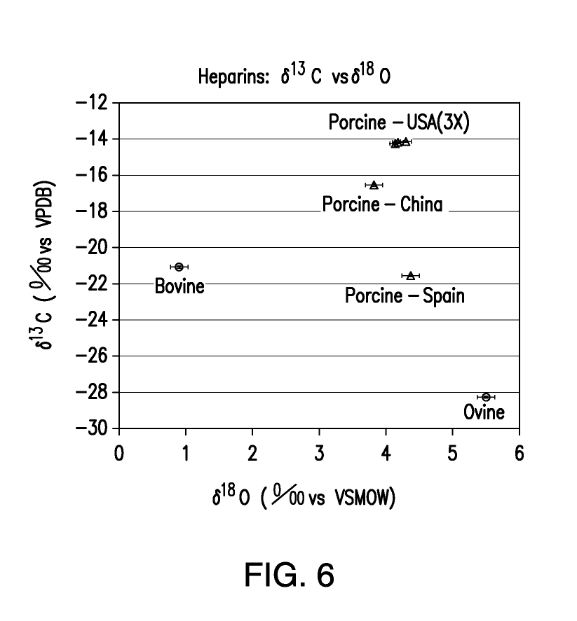

Four bivariate plots of the stable-isotopic results collected on the heparin samples are given in FIGS. 6-9. to show the interrelationship of the isotopic results and their groupings.

The δX-δ¹³C Bivariate Plots.

Although there are 12 such plots, PCA (FIG. 5) shows that the four paired δX−δ¹⁸O (where X=¹³C, ¹⁵N, ¹⁸O, and ³⁴S) values can be used for the analysis. These results are indicated in FIG. 5, where the correlation between the stable isotopes can be deduced from the loadings, as well as the ability for each of the five stable isotopes to differentiate between the heparin samples. The eight other optional plots complete the set of twelve.

Four bivariate isotopic plots of the paired δX-δ¹⁸O values of the three (ovine, bovine, porcine) heparin samples are shown in FIGS. 6-9. For reference, the typical standard errors of the isotopic measurements are shown in the graphs and are given in Table 2.

Preliminary Assessment of Digestive and Environmental Effects on Isotopic Values

Thirty-five measurements of the carbon-(δ¹³C), nitrogen-(δ¹⁵N), and sulfur-(δ³⁴S) isotope ratios of five samples of animal-derived heparins, and presumably other biologic molecules, readily allow differentiation of the samples into groups and/or subgroups based on their isotopic provenance.

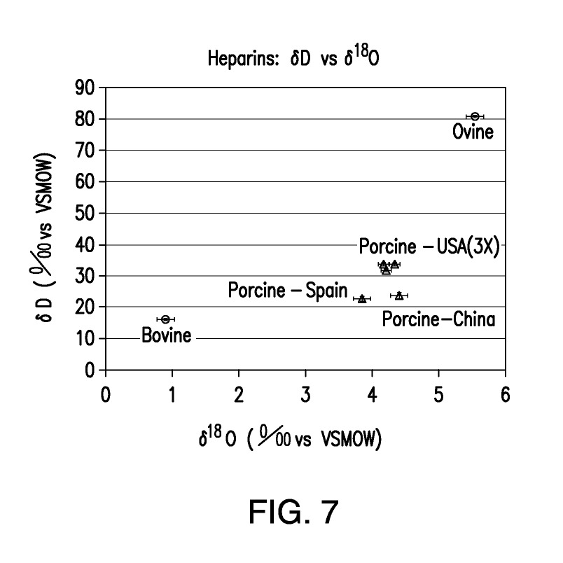

In particular, the stable-isotopic analyses revealed that (i) stable-isotopic measurements of biologic molecules (MW˜10-15 kDa) were feasible; (ii) in bivariate plots, the δ¹³C vs δ¹⁸O plot (FIG. 6.) reveals a well-defined relationship for source differentiation, separating USA-porcine heparin from non-USA heparin; (iii) the δD vs δ¹⁸O plot (FIG. 7.) reveals the relationship for source differentiation based on the hydrologic-environmental isotopes of water (D/H. and ¹⁸O/¹⁶O; and (iv) the δ¹⁵N vs δ¹⁸O and δ³⁴S vs δ¹⁸O relationships are similar, revealing the food sources for the heparin producers.

Finally, a stable-isotopic process study (e.g., Jasper et al., 2007) should reveal the connectedness of the isotopic connections and synthetic pathways for the production.

### Example 2

### Stable-Isotopic Analysis of Biologic Samples

The methodology of Example 1 can be applied to a wide variety of biological product. Biologics are becoming an ever increasingly important class of compounds expected to account for approximately 17% of the total global spending in medicines by 2016 (Van Arnum P., (2013) Tracking Growth in Biologics. Pharmaceutical Technology Vol. 37, Issue 2.), which is incorporated by reference herein in its entirety. Examples of other biologics that can be used include [Drug Name (Trade Name)] that have been approved or are currently in development include: abciximab (ReoPro), adalimumab (Humira), ado-trastuzumab emtansine (Kadcyla), alemtuzumab (Campath-1H; Lemtrada; MabCampath), alirocumab (in clinical trials), basiliximab (Simulect), belimumab (Benlysta), bevacizumab (Avastin), blinatumomab (Blincyto), bococizumab (PF-04950615—in clinical trials), brentuximab vedotin (Adcetris), canakinumab (Ilaris), catumaxomab (Removab), certolizumab pegol (Cimzia), cetuximab (Erbitux), daclizumab (Zenapax), Dekavil (Pfizer—in clinical trials), denosumab (Prolia), dinutuximab (Unituxin—in clinical trials), eculizumab (Soliris), efalizumab (Raptiva), emactuzumab (Lemtrada—in clinical trials), etanercept (Enbrel), etrolizumab (RG7413—in clinical trials), evolocumab (in clinical trials), gantenerumab (RG1450—in clinical trials), gemtuzumab ozogamicin (Mylotarg), golimumab (Simponi), ibritumomab tiuxetan (Zevalin), infliximab (Remicade), inotuzumab (ozogamicin—in clinical trials), insulin glargine (Lantus), interferon beta-1a (Avonex), ipilimumab (Yervoy), lampalizumab (RG7417—in clinical trials), lebrikizumab (RG3637—in clinical trials), lifastuzumab vedontin (RG7599—in clinical trials), mepolizumab (in clinical trials), motavizumab (Numax), muronomab-CD3 (Orthoclone OKT3), natalizumab (Tysabri), necitumumab (in clinical trials), nivolumab (Obdivo), obinutuzumab (Gazyva—in clinical trials), ocrelizumab (RG1594—in clinical trials), ofatumumab (Arzerra), omalizumab (Xolair), palivizumab (Synagis), panitumumab (Vectibix), PD-0360324 (Pfizer—in clinical trials), pegfilgrastim (Neulasta), pembrolizumab (Keytruda), pertuzumab (Perjeta—in clinical trials), PF-03446962 (Pfizer—in clinical trials), PF-04236921 (Pfizer—in clinical trials), PF-05082566 (Pfizer—in clinical trials), PF-05230907 (Pfizer—in clinical trials), PF-05236812 (AAV-003—in clinical trials), PF-05280602 (Pfizer—in clinical trials), PF-05285401 (Pfizer—in clinical trials), PF-06252616 (Pfizer—in clinical trials), PF-06263507 (Pfizer—in clinical trials), PF-06342674 (Pfizer—in clinical trials), PF-06480605 (Pfizer—in clinical trials), PF-06647263 (Pfizer—in clinical trials), PF-06650808 (Pfizer—in clinical trials), PF-00547659 (Pfizer—in clinical trials), polatuzumab vedotin(RG7596—in clinical trials), ponezumab (PF-04360365—in clinical trials), ramucirumab (Cyramza), ranibizumab (Lucentis), raxibacumab (ABThrax), rituximab (Rituxan; MabThera), secukinumab (Cosentyx), siltuximab (Sylvant), Tanezumab (Pfizer—in clinical trials), tocilizumab (Actemra—in clinical trials), tositumomab-I-131 (Bexxar), trastuzumab (Herceptin), ustekinumab (Stelara), vanucizumab (RG7221—in clinical trials), vedolizumab (Entyvio). The present invention is also applicable to a combination of biologics.

### REFERENCES

The following references are incorporated by reference herein in their entirety.
“Biologics Research Pushing Frontiers of Science With More Than 900 Medicines and Vaccines in Development.” Medicines in Development: Biologics (2013) Pharmaceutical Research and Manufacturers of America. 2013 Report 1-87
Fogel, M. L. (2010). Variations in abundances of nitrogen isotopes in Nature. In: Encyclopedia of Mass Spectrometry, Vol. 5. Elsevier, Oxford UK (preprint).
Fritz, P. and Fontes, J. Ch. (1981). Handbook of Environmental Isotope Geochemistry; 1. The Terrestrial Environment. Elsevier, Amsterdam, p. 394.
Fu L, Li G, Yang B, Onishi A, Li L, Sun P, Zhang F, Linhardt R J. (2013). Structural characterization of pharmaceutical heparins prepared from different animal tissues. Journal of Pharmaceutical Sciences 102:1447-1457)
Habfast, K. (1997). Advanced Isotope Ratio Mass Spectrometry I: Magnetic Isotope Ratio Mass Spectrometers. In: Modern Isotope Ratio Mass Spectrometry, I. T. Platzner, Ed., J. Wiley and Sons, Ltd., New York, pp. 11-82.
Jasper, J. P. (1999) The increasing use of stable isotopes in the pharmaceutical industry. Pharm. Tech. 23(10):106-114.
Jasper, J. P. (2001) Quantitative estimates of precision for molecular isotopic measurements. Rap. Comm. Mass Spec. 15:1554-1557.
Jasper, J. P. (2004) Pharmaceutical security: Using stable isotopes to authenticate pharmaceutical materials. Tablets and Capsules 2(3):37-42.
Jasper, J. P., R. C. Lyon, and L. E. Weaner (2005) Stable isotopes provide a new PAT [Process Analytical Technology] tool. Pharm. Mfg. 4(5):28-33.
Jasper, J. P., Weaner, L. E., and Hayes, J. M. (2007). Process Patent Protection: Characterizing synthetic pathways by stable-isotopic measurements. Pharm. Technol. 31(3):68-73.
Jasper, J. P., Pavane, M., Eyler, D., Sharma, I. and Lee, A. (2012) Process Patent Protection: Protecting intellectual property via natural-abundance stable isotopes. Am. Intel. Prpty. Law. Assoc. webinar (Nov. 13, 2012).
Jasper. J. P., Zhang F., Poe R. B., Linhardt R. J. (2015) Stable Isotopic Analysis of Porcine, Bovine, and Ovine Heparins. Journal of Pharmaceutical Sciences. 104:457-463.
Lawrence, Stacy & Lahteenmaki, Riku (2014). Public biotech 2013—the numbers. Top-ten-selling biologic drugs of 2013. Nature Biotechnology 32, 626-632 (2014). doi: 10.1038/nbt2949
Smith B. N. and S. Epstein (1971). Two categories of ¹³C/¹²C ratios for higher plants. Plant Physiol. 47:38-384.
Stone, Kathlyn. The Top 10 Biologic Drugs. (2013) About: Money: Pharma.
http://pharma.about.com/od/SalesandMarketing/tp/The-Top-10-Biologic-Drugs.htm
Top 8 blockbuster biologicals 2013. (2014) Genetic Engineering News. Generics and Biosimilars Initiative (GaBI) http://www.gabionline.net/Biosimilars/General/Top-8-blockbuster-biologicals-2013. Posted Jun. 6, 2014
Van Arnum P., (2013) Tracking Growth in Biologics. Pharmaceutical Technology Vol. 37, Issue 2.
Jasper, J. P., Stable Isotopic Identification and Method for Identifying Products by Isotopic Composition. (U.S. Pat. No. 7,323,341).
Jasper, J. P., Tracing Processes Between Precursors and Products by Utilizing Isotopic Relationships. (U.S. Pat. No. 8,367,414 B2).

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

1. A method for objectively identifying a biological product, comprising:
obtaining isotopic data from elements present in said biological product;
providing a mathematical array that includes the isotopic data, the mathematical array being fixed in a readable form, said readable form with said mathematical array fixed thereon being an identification of said biological product;
and wherein the isotopic data does not include data obtained from a taggant.

2. A method for objectively identifying a biological product, comprising:
obtaining isotopic data from elements present in said biological product;
providing a mathematical array that includes the isotopic data, the mathematical array being fixed in a readable form, said readable form with said mathematical array fixed thereon being an identification of said biological product.

3. A method for constructing an isotopic process profile for a biological product made using a known synthetic process, comprising: obtaining a first isotopic composition profile for elements present in the biological product and a second isotopic composition profile for elements present in one or more starting materials used to make the biological product; determining isotopic fractionation values for one or more reaction steps in the known synthetic process; and providing a database that includes a plurality of data selected from the group consisting of (i) the first isotopic composition profile for the biological product, (ii) the second isotopic composition profile for one or more starting materials used to make the biological product, and (iii) isotopic fractionation values for one or more reaction steps in the known synthetic process; wherein the database is an isotopic process profile of the biological product.

4. A method for determining whether a biological product of undefined origin was made by a first known synthetic process, comprising: obtaining a first isotopic composition profile for elements present in the biological product; providing fractionation information regarding the first known synthetic process, the starting materials used to make the biological product, or both; and inferentially determining whether the biological product of undefined origin was made by the first known synthetic process by comparing the first isotopic composition profile to the information.

5. A method for monitoring process quality of a biological synthesis process for a biological product, comprising: defining an acceptable range of isotopic abundance for elements present in the biological product at an intermediate point or an end point in the biological synthesis process for at least one stable isotope, the acceptable range encompassing isotopic abundance values that exist when the process is proceeding in an acceptable manner; periodically extracting samples from the biological synthesis process at the intermediate point or the end point; measuring the actual isotopic abundance for the at least one stable isotope in the samples; and comparing the actual isotopic abundance to the acceptable range to determine whether the biological synthesis process is proceeding in an acceptable manner.

6. A system for monitoring process quality of a biological synthesis process for a biological product, comprising: a sample extraction device operable to periodically obtain samples from a process stream for the biological synthesis process at an intermediate point or an end point in the process; a measuring instrument operable to receive the samples from the extraction device and determine actual isotopic abundance information for one or more isotopes for elements present in the samples; and a computer processor operable to store and display the isotopic abundance information.

7. A method for making a new biological product batch for a biological product that has a highly-specific isotopic composition profile for elements present in the biological product different than a previously-made biological product batch with the same molecular content, comprising adjusting at least one aspect of the manufacturing process for the biological product in a manner selected from the group consisting of (i) selecting a starting material having a different isotopic composition profile for elements present in the starting material, (ii) identifying a chemical reaction in the process that has an isotope effect, and halting the reaction at a different stage short of completion, (iii) identifying a chemical reaction in the process that has an isotope effect, and making the limiting reagent one that is not used to derive the isotopic composition profile of the biological product, (iv) altering the amount of the limiting reagent that is available for reaction, and (v) mixing into the product an excipient having a different isotopic composition profile for elements present in the excipient.

8. A method according to claim 1 wherein the biological product has a molecular weight of at least about 1000 daltons.

9. A method according to claim 8 wherein the biological product has a molecular weight of at least about 1000 daltons to about 2,000,000 daltons.

10. A method according to claim 8 wherein the biological product has a molecular weight of at least about 1000 daltons to about 1,000,000 daltons.

11. A method according to claim 8 wherein the biological product has a molecular weight of at least about 1000 daltons to about 500,000 daltons.

12. A method or system according to claim 8 wherein the biological product has a molecular weight of at least about 1000 daltons to about 100,000 daltons.

13. A method according to claim 1, wherein the biological product is selected from abciximab (ReoPro), adalimumab (Humira), ado-trastuzumab emtansine (Kadcyla), alemtuzumab (Campath-1H; Lemtrada; MabCampath), alirocumab, basiliximab (Simulect), belimumab (Benlysta), bevacizumab (Avastin), blinatumomab (Blincyto), bococizumab (PF-04950615), brentuximab vedotin (Adcetris), canakinumab (Ilaris), catumaxomab (Removab), certolizumab pegol (Cimzia), cetuximab (Erbitux), daclizumab (Zenapax), Dekavil (Pfizer), denosumab (Prolia), dinutuximab (Unituxin), eculizumab (Soliris), efalizumab (Raptiva), emactuzumab (Lemtrada), etanercept (Enbrel), etrolizumab (RG7413), evolocumab, gantenerumab (RG1450), gemtuzumab ozogamicin (Mylotarg), golimumab (Simponi), heparin (Lipo-Hepin/Liquaemin/Panheparin), ibritumomab tiuxetan (Zevalin), infliximab (Remicade), inotuzumab (ozogamicin), insulin glargine (Lantus), interferon beta-1a (Avonex), ipilimumab (Yervoy), lampalizumab (RG7417), lebrikizumab (RG3637), lifastuzumab vedontin (RG7599), mepolizumab, motavizumab (Numax), muronomab-CD3 (Orthoclone OKT3), natalizumab (Tysabri), necitumumab, nivolumab (Obdivo), obinutuzumab (Gazyva), ocrelizumab (RG1594), ofatumumab (Arzerra), omalizumab (Xolair), palivizumab (Synagis), panitumumab (Vectibix), PD-0360324 (Pfizer), pegfilgrastim (Neulasta), pembrolizumab (Keytruda), pertuzumab (Perjeta), PF-03446962 (Pfizer), PF-04236921 (Pfizer), PF-05082566 (Pfizer), PF-05230907 (Pfizer), PF-05236812 (AAV-003), PF-05280602 (Pfizer), PF-05285401 (Pfizer), PF-06252616 (Pfizer), PF-06263507 (Pfizer), PF-06342674 (Pfizer), PF-06480605 (Pfizer), PF-06647263 (Pfizer), PF-06650808 (Pfizer), PF-00547659 (Pfizer), polatuzumab vedotin (RG7596), ponezumab (PF-04360365), ramucirumab (Cyramza), ranibizumab (Lucentis), raxibacumab (ABThrax), rituximab (Rituxan; MabThera), secukinumab (Cosentyx), siltuximab (Sylvant), Tanezumab (Pfizer), tocilizumab (Actemra), tositumomab-I-131 (Bexxar), trastuzumab (Herceptin), ustekinumab (Stelara), vanucizumab (RG7221), vedolizumab (Entyvio), and mixtures thereof.

14. A method according to claim 1, wherein the biological product is heparin.

15. A method according to claim 14, wherein the heparin is mammalian heparin.

16. A method according to claim 15, wherein the mammalian heparin is selected from human heparin, bovine heparin, ovine heparin, porcine heparin, and mixtures thereof.

17. A method according to claim 15, wherein the mammalian heparin is human heparin.

18. A method according to claim 15, wherein the mammalian heparin is bovine heparin.

19. A method according to claim 15, wherein the mammalian heparin is ovine heparin.

20. A method according to claim 15, wherein the mammalian heparin is porcine heparin.

21. A method according to claim 1 wherein the elements are selected from elements that have two or more isotopes.

22. A method according to claim 21 wherein the elements are selected from hydrogen, carbon, nitrogen, oxygen, sulfur, chlorine, bromine, and combinations thereof.

23. A method according to claim 22 wherein the isotopes are stable isotopes.

24. A method according to claim 23 where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

25. A method according to claim 24 wherein the isotope ratios are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

26. A method according to claim 24 wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

27. A method according to claim 26 wherein the isotope ratio is ²H/¹H.

28. A method according to claim 26 wherein the isotope ratio is ¹³C/¹²C.

29. A method according to claim 26 wherein the isotope ratio is ¹⁵N/¹⁴N.

30. A method according to claim 26 wherein the isotope ratio is ¹⁸O/¹⁶O.

31. A method according to claim 26 wherein the isotope ratio is ³⁴S/³²S.

32. A method according to claim 26 wherein the isotope ratio is ³⁷Cl/³⁵Cl.

33. A method according to claim 26 wherein the isotope ratio is ⁸¹Br/⁷⁹Br.

34. A method according to any claim 1 wherein the isotopic data is intrinsic isotopic data.

35-47. (canceled)

48. A method according to claim 3 wherein the isotopic composition profile is an intrinsic isotopic composition profile.

49. A method according to claim 5 wherein the isotopic abundance information is intrinsic isotopic abundance information.
